(19) United States
(12) Patent Application Publication
(10) Pub. No.: US 2014/0067274 A1
(43) Pub. Date: Mar. 6, 2014

(54) METHOD FOR PROCESSING PHYSIOLOGICAL SIGNAL

(71) Applicant: Dong Hwa LEE, Yongin-si (KR)

(72) Inventor: Dong Hwa LEE, Yongin-si (KR)

(21) Appl. No.: 13/962,930

(22) Filed: Aug. 8, 2013

(30) Foreign Application Priority Data
Sep. 6, 2012 (KR) 10-2012-0098774

(51) Int. Cl. A61B 5/00

(52) U.S. Cl. CPC A61B 5/7239
USPC 702/19; 600/364

(57) ABSTRACT

Disclosed herein is a method for processing a physiological signal to measure physiological characteristics of a living organism. The method includes: (a) dividing P₁′(t), which is obtained by differentiating one signal function P₁(t) between two signal functions having different wavelength bands and representing the physiological characteristics of the living organism, by P₂′(t) obtained by differentiating the other signal function P₂(t); and (b) obtaining a constant b of the following function n₁(t) and a cycle of the following function s₁(t) through Math Formula 1:

(P₁′(t)/P₂′(t))|_{t=t1,t2,t3, . . .} =k,
(where P₁(t)=s₁(t)+n₁(t), P₂(t)=s₂(t)+n₂(t), as₁(t)=s₂(t), bn₁(t)=n₂(t), a, b and k are constants, t is time, and n₁(t) and n₂(t) are signal functions due to noise when measuring the physiological signal).

### BACKGROUND

1. Technical Field

The present invention relates to a method for processing a physiological signal, and more particularly, to a method for processing a physiological signal in which, when a non-invasive method is used to measure oxygen saturation, pulse or similar biological characteristics (i.e., physiological characteristics), a characteristic constant and cycle of a signal for checking the biological characteristics can be readily obtained, whereby the biological characteristics can be rapidly and easily determined.

2. Description of the Related Art

In general, a signal processor processes a physiological signal based on various physiological characteristics, for example, blood gas saturation such as oxygen saturation or the like, blood pressure, heart rate, electrocardiogram, and the like, and obtains a physiological signal by extracting or removing one of a main signal and a noise signal from a composite signal including the main and noise signal.

For instance, the composite signal includes a main signal component containing desired data on a user, and a noise signal component generated by noise. At this time, if the noise signal occupies a frequency spectrum different from that of the main signal, it is possible to remove the noise signal or extract the main signal from the whole signal through existing filtering methods such as low pass filtering, band pass filtering, high pass filtering, etc. However, the main signal and the noise signal may have an overlapping frequency spectrum.

In this case, the existing filtering methods cannot effectively extract the main signal or remove the noise signal. Further, if information about one of the two signal components is sufficient, a correlation canceller such as an adaptive noise canceller may be used to extract the signal component. However, in general, information about the signals is not sufficient.

Accordingly, in the case of physiological monitoring, i.e., in monitoring of physiological characteristics, it is not easy to determine information about the main signal component and the noise signal component. Physiological monitoring generally uses a measured signal derived from a biological system such as a human body in order to monitor physiological characteristics.

The subjects of the measurement in physiological monitoring systems generally include various types such as electrocardiographs, blood pressure, blood gas saturation (e.g.: oxygen saturation or the like), capnographs, monitoring of different blood components, heart rate, respiratory rate, etc. As an example of oxygen saturation which is a form of blood gas saturation, there is arterial oxygen saturation.

Measurements of such various physiological characteristics may be achieved using energy attenuation through an inspection medium such as a finger in the form of selected energy. Among these, blood gas monitoring is an example of physiological monitoring based on the measurement of energy attenuation due to biological tissues or materials.

In blood gas monitoring, light for measuring a physiological signal is emitted to an inspection medium, for example, a patient's finger, and an attenuation characteristics of the light are measured over time. At this time, an output signal of a blood gas monitor susceptible to flow of arterial blood includes a signal having a waveform reflecting an arterial pulse of a patient.

Examples of such physiological monitoring technology are disclosed in patent documents, such as U.S. Pat. Nos. 6,002,952 and 5,490,505. In the case of using an inspection medium such as a finger to carry out physiological monitoring, venous blood, movement of the finger and similar noise factors may cause irregular energy attenuation.

In U.S. Pat. No. 6,002,952, FIG. 1 shows a graph of a signal waveform (i.e., a waveform of the main signal) according to regular energy attenuation, and FIG. 2 shows a graph of a signal waveform according to practical irregular energy attenuation, i.e., considering a noise component.

However, if an irregular signal waveform, due to venous blood, patient movement, and the like, is detected as described above, complicated operations and processes are needed to extract a main signal from an irregular signal waveform. Therefore, it is difficult to extract information of the main signal, i.e., the cycle and characteristic constant of the main signal and to output the physiological characteristics based on the information.

### BRIEF SUMMARY

The present invention has been conceived to solve the above problems, and it is an aspect of the invention to provide a method for processing a physiological signal, in which information of a physiological signal measured by (i.e., input by) a detector, particularly, characteristics of a main signal can be obtained without noise signal through a simple process.

In accordance with one aspect of the present invention, a method for processing a physiological signal to measure physiological characteristics of a living organism includes: (a) dividing P₁′(t), which is obtained by differentiating one signal function P₁(t) between two signal functions having different wavelength bands and representing the physiological characteristics of the living organism, by P₂′(t) obtained by differentiating the other signal function P₂(t); and (b) obtaining a constant b of the following function n₁(t) and a cycle of the following function s₁(t) through Math Formula 1 as follows:

(P₁′(t)/P₂′(t))|_{t=t1,t2,t3, . . .} =k, [Math Formula 1]

(where P₁(t)=s₁(t)+n₁(t), P₂(t)=s₂(t)+n₂(t), as₁(t)=s₂(t), bn₁(t)=n₂(t), a, b and k are constants, t is time, and n₁(t) and n₂(t) are signal functions due to noise when measuring the physiological signal).

The step of obtaining a constant b of the function n₁(t) and a cycle of the function s₁(t) comprises obtaining the constant b of the function n₁(t) and the cycle of the function s₁(t) by setting the value k of Math Formula 1 such that a variable t allowing the function P₁′(t)/P₂′(t) to have the same value can satisfy a condition of including a value repeated at a regular cycle.

The step of obtaining a constant b of the function n₁(t) and a cycle of the function s₁(t) may comprise obtaining the constant b of the function n₁(t) and the cycle of the function s₁(t) by setting the value k of Math Formula 1 so as to satisfy a condition that intersecting points between a transverse line (k) parallel to an axis (t) of abscissa of the function P₁′(t)/P₂′(t) and the function P₁′(t)/P₂′(t) include intersecting points repeated at regular intervals with at least one intersecting point therebetween.

The step of obtaining a constant b of the function n₁(t) and a cycle of the function s₁(t) may comprise obtaining the constant b of the function n₁(t) and the cycle of the function s₁(t) by setting the value k of Math Formula 1 so as to satisfy a condition that intersecting points between a transverse line (k) parallel to an axis (t) of abscissa of the function P₁′(t)/P₂′(t) and the function P₁′(t)/P₂′(t) include intersecting points repeated in a certain pattern.

The step of obtaining a constant b of the function n₁(t) and a cycle of the function s₁(t) may comprise obtaining the constant b of the function n₁(t) and the cycle of the function s₁(t) by setting the value k of Math Formula 1 so as to satisfy a condition that intersecting points between a transverse line (k) parallel to an axis (t) of abscissa of the function P₁′(t)/P₂′(t) and the function P₁′(t)/P₂′(t) include intersecting points of a preset pattern.

The Math Formula 1 used in extracting the constant b of the function n₁(t) and the cycle of the function s₁(t) can be obtained based on that s₁′(t) obtained by differentiating the function s₁(t) is 0 (zero).

The physiological characteristics may include pulse oxygen saturation, and may be measured by an infrared signal and a red light signal.

After the constant b is extracted in step (b), it is possible to extract a reference waveform of the signal function s₁(t) through Math Formula 4 as follows:

P₁(t)−P₂(t)/b=s₁(t)+n₁(t)−(as₁(t)+bn₁(t))/b=(1−a/b)s₁(t). [Math Formula 4]

According to the present invention, the period of data without noise can be obtained through a simple process and detection process of the physiological characteristics can be simplified.

### BRIEF DESCRIPTION OF THE DRAWINGS

The above and other aspects, features, and advantages of the invention will become apparent from the detailed description of the following exemplary embodiments in conjunction with the accompanying drawings, in which:

FIG. 1 is a graph schematically showing an exemplary waveform of an ideal signal (a main signal) free from any noise factors;

FIG. 2 is a graph schematically showing an example waveform of a practical signal (a composite signal) with noise;

FIG. 3 is a flowchart showing a method for processing a physiological signal according to one embodiment of the present invention;

FIG. 4 is a graph for explaining a method of extracting a cycle of a main signal in the method for processing a physiological signal according to the embodiment of the present invention; and

FIG. 5 is a graph for explaining a method of extracting a reference pattern of a main signal in the method for processing a physiological signal according to the embodiment of the present invention.

### DETAILED DESCRIPTION

Now, exemplary embodiments of the invention will be described in detail with reference to the accompanying drawings. In describing the exemplary embodiments, like terms and numerals refer to like components throughout the specification and the accompanying drawings, and thus repeated descriptions thereof will be omitted.

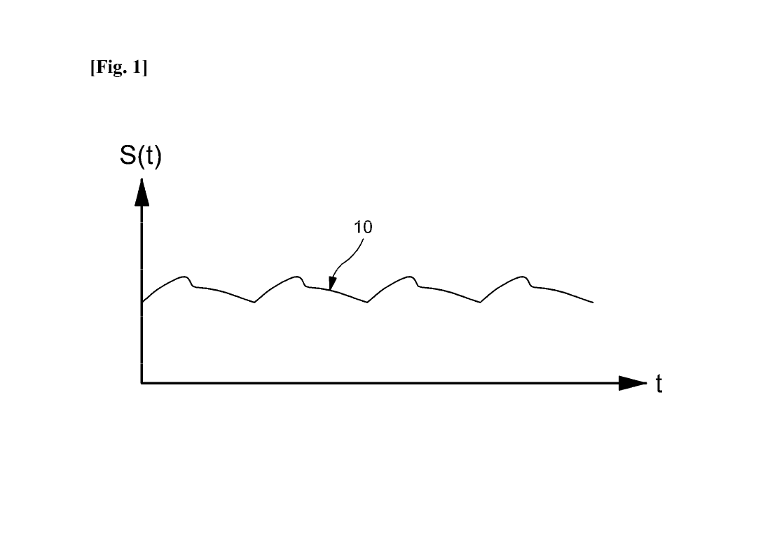

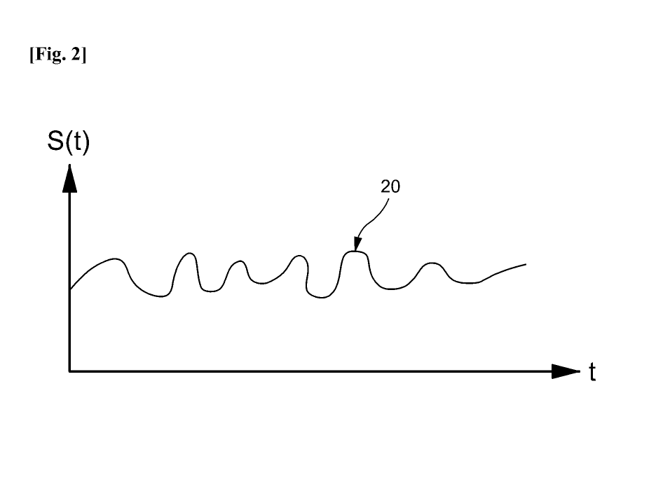

Referring to FIGS. 1 and 2, when a non-invasive method is used to measure physiological characteristics, a waveform 10 of an ideal signal (i.e., a main signal) at a regular cycle is detected, as shown in FIG. 1, if there is no noise factor. On the other hand, if a noise factor is present, an irregular waveform 20 of a signal in which noise signal is mixed with the main signal is detected, as shown in FIG. 2.

The irregular signal waveform 20 shows a result of adding a noise factor to the ideal signal waveform 10. If such a noise factor is present in measurement of the physiological characteristics of a living organism, it can be difficult to distinguish the main signal having data of the physiological characteristics even though the noise factor is small.

To measure the physiological characteristics, signals detected by a sensor such as a pulse oximeter or the like, that is, measured signals, include two measured signals. The two measured signals may belong to different wavelength bands, and thus the physiological characteristics of the living organism may be determined through the two signal components.

Each of the two measured signals, that is, the signals 20 (measured signals or input signals) measured by the sensor, includes a main signal 10 and a noise signal. Here, the main signal is an ideal measured signal when no noise factor is present, and the noise signal is a signal measured due to a noise factor, such as movement of a patient, venous blood, etc.

As detailed examples of the two measured signals, there are signals used in measuring the oxygen saturation, which includes infrared signals measured using infrared (IR) light and red light signals measured using red light.

Here, a device (i.e., a sensor) for measuring the oxygen saturation of arterial blood through a non-invasive method is called a pulse oximeter, which obtains blood oxygen saturation, which is an indicator of the amount of hemoglobin present in the arterial blood. The oxygen saturation is information utilized in heart output or pulmonary function evaluation, organ specific perfusion or cardiovascular state evaluation, hypoxia diagnosis, etc., which is applied to various fields including not only general medicine but also sports science.

The pulse oximeter includes two light emitters for emitting two kinds of light to a body (e.g., a finger) of a patient (e.g., an examinee), and a detector such as a photodiode, in which two light emitters are installed, for example, a red light emitting diode (LED) for emitting red light and an infrared LED for outputting infrared light may be provided.

Further, each of the two measured signals exhibiting the foregoing oxygen saturation or similar physiological characteristics is a composite signal wherein a main signal and noise signal are mixed, as described above. Hereinafter, a function representing the waveform 10 of the main signal will be denoted by s(t), a function representing the waveform 20 of the noise signal (i.e., a signal with noise in measurement of the physiological signals) will be denoted by n(t), and a function of the measured signals (i.e., the composite signals) in this embodiment will be denoted by P(t) which is represented by the sum of the functions s(t) and n(t).

In more detail, one of two signals, i.e., the two measured signals representing the physiological characteristics of a living organism such as a human body may be represented by the function P₁(t), and the other signal may be represented by the function P₂(t). The functions P₁(t) and P₂(t) are not limited to a certain signal. In other words, if one of the two signals is P₁(t), the other signal is P₂(t).

Here, P₁(t) and P₂(t) are defined as follows.

P₁(t)=s₁(t)+n₁(t), P₂(t)=s₂(t)+n₂(t)

In P₁(t) and P₂(t), as₁(t)=s₂(t) and bn₁(t)=n₂(t), and a and b are constants. Also, P₁(t) and P₂(t) are time dependent functions, in which a variable t refers to time.

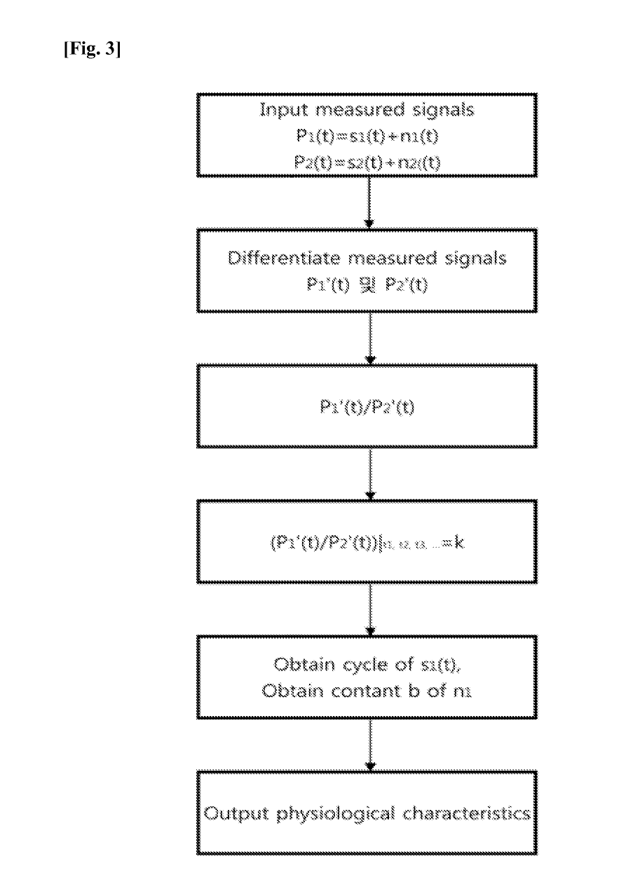

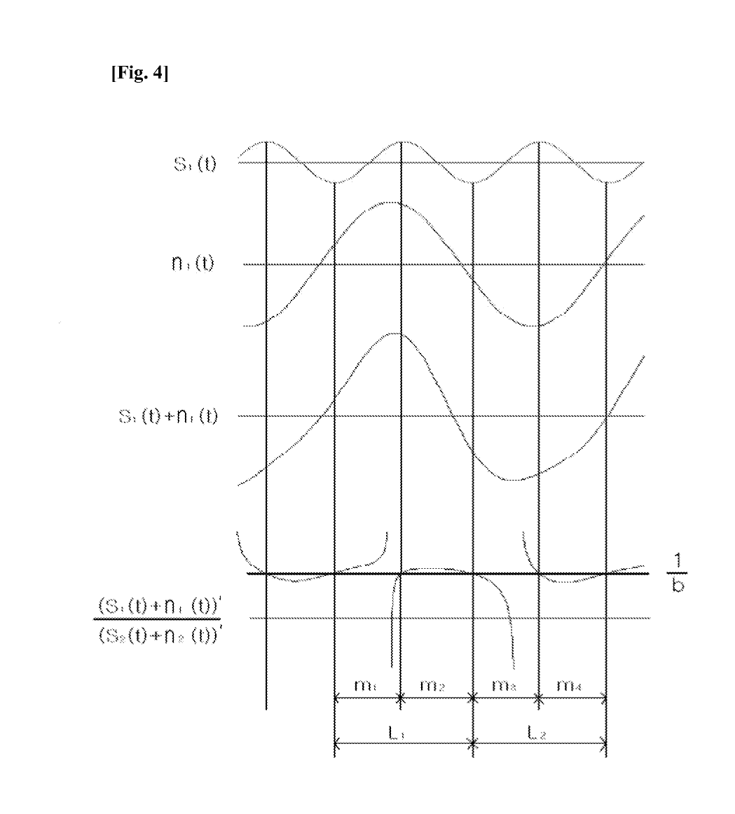

Next, referring to FIGS. 3 and 4, as one embodiment of the method for processing a physiological signal according to the invention, a method of obtaining information about the main signal containing data of physiological characteristics, in particular, an exemplary method of extracting a cycle of the main signal will be described.

In this embodiment, the method for processing a physiological signal for measuring physiological characteristics of a living organism includes: step (a) dividing P₁′(t), which is obtained by differentiating one signal function (hereinafter, referred to as P₁(t) between two signal functions (P(t)) having different wavelength bands and representing the physiological characteristics of the living organism, by P₂′(t) obtained by differentiating the other signal function (hereinafter, referred to as P₂(t)); and step (b) obtaining a constant b of n₁(t) and the cycle of the function s₁(t). Here, the step (b) is processed using Math Formula 1 as follows.

(P₁′(t)/P₂′(t))|_{t=t1,t2,t3, . . .} =k. [Math Formula 1]

That is, the function obtained in step (a) may be represented by Math Formula 2 as follows:

(P₁′(t)/P₂′(t)=(s₁′(t)+n₁′(t))/(s₂′(t)+n₂′(t)). [Math Formula 2]

Thus, the Math Formula 2 may be represented by (s₁′(t)+n₁′(t))/(as₁′(t)+bn₁′(t)), and if s₁′(t) obtained by differentiating the function s₁(t) is 0 (Zero), the value of the Math Formula 2 becomes (1/b), and (1/b)=k is established under the same conditions.

A signal processor, which processes a physiological signal caused by the physiological characteristics such as oxygen saturation and the like, generates the function P₁(t) from one signal (e.g., an infrared signal) of the two measured signals input to the signal processor and generates the function P₂(t) from the other signal (e.g., red light signal). It should be understood that the functions P₁(t) and P₂(t) are not limited to the above description. Alternatively, the function P₁(t) may be a function for a red light signal and the function P₂(t) may be a function for an infrared signal.

Further, in measurement of the pulse oxygen saturation, s₁(t) and s₂(t) may be signal functions by arterial blood, and n₁(t) and n₂(t) may be signal functions by noise factors such as venous blood or the like.

Next, P₁(t) and P₂(t) are respectively differentiated to obtain the functions P₁′(t) and P₂′(t), and one of the functions P₁′(t) and P₂′(t) is used to divide the other function, thereby obtaining the Math Formula 2 described above.

Further, to extract the cycle of the function s₁(t) at the step (b), if k of the Math Formula 1 is extracted such that the values of variable t allowing the function P₁′(t)/P₂′(t) defined in Math Formula 2 to have the same value can satisfy the condition of including a value repeated in a constant pattern or cycle, a constant b of n₁(t) and the cycle of the function s₁(t) can be obtained.

Referring to FIG. 4, a model for extracting the cycle of the function s₁(t) will be described in more detail. In a graph according to the function of Math Formula 2 having an axis of abscissa representing time t, if intersecting points between a virtual transverse line (k) and the graph of the Math Formula 2 include points repeated in a certain pattern when the virtual transverse line (k) is horizontally shifted in a vertical direction, an inverse of a value on the virtual transverse line (k) becomes k of Math Formula 1.

At this time, according to the Math Formula 2, the values t of the intersecting points between the virtual transverse line (k) and the function of the Math Formula 2 include the values t of coordinates (valleys and peaks of the function s₁(t)) where s₁′(t) obtained by differentiating the function s₁(t) becomes 0 (zero), and thus a distance between the valleys or between the peaks of the function s₁(t) is the cycle of the function s₁(t).

Therefore, if the intersecting points between the virtual transverse line (k) and the graph of Math Formula 2 include points repeated at regular intervals with at least one point therebetween, or if the transverse line (k) is determined such that the intersecting points between the virtual transverse line (k) and the graph of Math Formula 2 can include a pair of points (e.g., one corresponding to the valley of the function s₁(t) and the other corresponding to the peak of the function s₁(t)) repeated at regular intervals, and the cycle of the points repeated at regular intervals with at least one point therebetween or the cycle of a pair of points repeated at regular intervals is extracted, the cycle of the points becomes the cycle of the function s₁(t).

In more detail, with reference to FIG. 4, if the position of the transverse line (k) is determined by shifting the transverse line (k) up or down such that the intersecting points between the transverse line (k) and the function of Math Formula 2 can have points repeated at regular intervals (L₁=L₂= . . . ), L₁ becomes the cycle of s₁(t).

Alternatively, if the position of the transverse line (k) is determined by shifting the transverse line (k) up or down such that the intersecting points between the transverse line (k) and the function of Math Formula 2 can have a pair of points repeated at regular intervals (m₁ or m₂), the cycle of repeating the pair of points becomes the cycle of s₁(t). Here, m₁=m₃= . . . and, m₂=m₄= . . . .

Further, if the position of the transverse line (k) is determined such that a ratio (m₁/m₂) of distances from one of the intersecting points between the transverse line (k) and the function of Math Formula 2 to a certain leftward point and to a certain rightward point can have periodic characteristics, it is possible to extract the constant b of the function n₁(t) and the cycle of the function s₁(t). That is, if (m₁/m₂)=(m₃/m₄)= . . . , it is possible to extract the cycle of s₁(t).

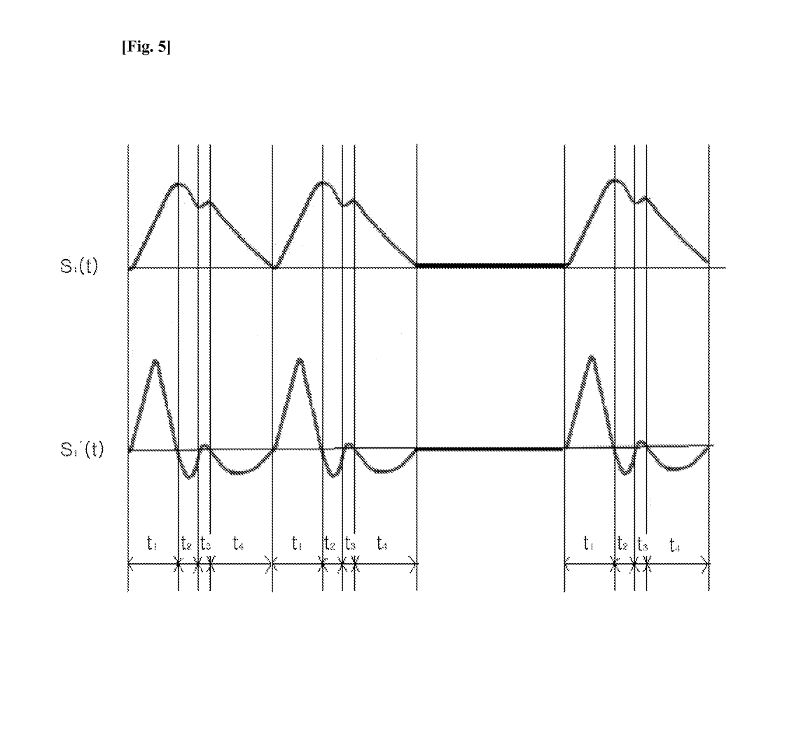

Further, as shown in the graph of FIG. 5, if the position of the transverse line (k) is determined by shifting the transverse line (k) up or down such that the intersecting points between the transverse line (k) and the function of Math Formula 2 can have points repeated in a reference pattern that is set to have a pattern of time t where a value of the differentiated signal s₁′(t) of one cycle including no noise n₁(t) becomes 0 (Zero), it is possible to extract the constant b of the function n₁(t) and the cycle of the function s₁(t).

In this embodiment, the method of extracting the cycle of the function s₁(t) is based on the fact that the value of Math Formula 2 becomes constant at a point (the valley and the peak of the function s₁(t)) where the function s₁′(t) becomes 0 (zero). It should be noted that the method of extracting the cycle may be modified in various ways without departing from the spirit of the present invention.

On the contrary, it is possible to extract the constant b and the cycle of the function s₁(t) or s₂(t) by the aforementioned method even though the function P₂′(t) is divided by the function P₁′(t) as shown in Math Formula 3 as follows. Since the functions s₁(t) and s₂(t) have the same cycle, it is possible to derive the constant b and the cycle of the main signal 10 of the two measured signals when one of the two functions is extracted.

(P₂′(t)/P₁′(t))=(1/k)=(s₂′(t)+n₂′(t))/(s₁′(t)+n₁′(t))=as₁′(t)+bn₁′(t))/(s₁′(t)+n₁′(t))=(s₂′(t)+n₂′(t))/((s₂′(t)/a)+(n₂′(t)/b) [Math Formula 3]

In addition, the cycles of the main signals are detected to ascertain the foregoing physiological characteristics and output the same.

Further, when the pattern of points, at which the value of the signal s₁′(t) including no noise n₁(t) is 0 (Zero), is known, Math Formula 1 may be used to obtain the constant b of the function n₁(t) and the cycle of the function s₁(t).

Further, after the constant b is extracted at step (b), a reference waveform of the signal s(t) may be extracted using Math Formula 4 as follows:

P₁(t)−P₂(t)/b=s₁(t)+n₁(t)−(as₁(t)+bn₁(t))/b=(1−a/b)s₁(t). [Math Formula 4]

In the method according to the invention, a signal cycle of desired data on a user, that is, data from which a noise component has been removed, can be extracted through a simple process, thereby simplifying the method for detecting physiological characteristics and thus reducing load applied to a signal processor for processing the physiological signal.

Further, the method according to the invention may reduce a period of time taken in processing the physiological signal, and load applied to devices constituting the signal processor, thereby reducing a heat generation rate of the biological signal sensor, such as a pulse oximeter, while extending lifespan of the devices and decreasing production costs.

Although some embodiments have been described herein, it will be understood by those skilled in the art that these embodiments are provided for illustration only, and that various modifications, changes, alterations and equivalent embodiments can be made without departing from the scope of the invention. Therefore, the scope and spirit of the invention should be defined only by the accompanying claims and equivalents thereof.

1. A method for processing a physiological signal to measure physiological characteristics of a living organism, comprising:
(a) dividing P₁′(t), which is obtained by differentiating one signal function P₁(t) between two signal functions having different wavelength bands and representing the physiological characteristics of the living organism, by P₂′(t) obtained by differentiating the other signal function P₂(t); and
(b) obtaining a constant b of the following function n₁(t) and a cycle of the following function s₁(t) through Math Formula 1.
(P₁′(t)/P₂′(t)))|_{t=t1,t2,t3, . . .} =k, [Math Formula 1]
(where P₁(t)=s₁(t)+n₁(t), P₂(t)=s₂(t)+n₂(t), as₁(t)=s₂(t), bn₁(t)=n₂(t), a, b and k are constants, t is time, and n₁(t) and n₂(t) are signal functions due to noise when measuring the physiological signal).

2. The method according to claim 1, wherein the step of obtaining a constant b of the function n₁(t) and a cycle of the function s₁(t) comprises obtaining the constant b of the function n₁(t) and the cycle of the function s₁(t) by setting the value k of Math Formula 1 such that a variable t allowing the function P₁′(t)/P₂′(t) to have the same value can satisfy a condition of including a value repeated at a regular cycle.

3. The method according to claim 1, wherein the step of obtaining a constant b of the function n₁(t) and a cycle of the function s₁(t) comprises obtaining the constant b of the function n₁(t) and the cycle of the function s₁(t) by setting the value k of Math Formula 1 so as to satisfy a condition that intersecting points between a transverse line (k) parallel to an axis (t) of abscissa of the function P₁′(t)/P₂′(t) and the function P₁′(t)/P₂′(t) include intersecting points repeated at regular intervals with at least one intersecting point therebetween.

4. The method according to claim 1, wherein the step of obtaining a constant b of the function n₁(t) and a cycle of the function s₁(t) comprises obtaining the constant b of the function n₁(t) and the cycle of the function s₁(t) by setting the value k of Math Formula 1 so as to satisfy a condition that intersecting points between a transverse line (k) parallel to an axis (t) of abscissa of the function P₁′(t)/P₂′(t) and the function P₁′(t)/P₂′(t) include intersecting points repeated in a certain pattern.

5. The method according to claim 1, wherein the step of obtaining a constant b of the function n₁(t) and a cycle of the function s₁(t) comprises obtaining the constant b of the function n₁(t) and the cycle of the function s₁(t) by setting the value k of Math Formula 1 so as to satisfy a condition that intersecting points between a transverse line (k) parallel to an axis (t) of abscissa of the function P₁′(t)/P₂′(t) and the function P₁′(t)/P₂′(t) include intersecting points of a preset pattern.

6. The method according to claim 1, wherein Math Formula 1 used in extracting the constant b of the function n₁(t) and the cycle of the function s₁(t) is obtained based on that s₁′(t) obtained by differentiating the function s₁(t) is 0 (zero).

7. The method according to claim 1, wherein the physiological characteristics comprise pulse oxygen saturation.

8. The method according to claim 1, wherein the physiological characteristics are measured by an infrared signal and a red light signal.

9. A method for processing a physiological signal to measure physiological characteristics of a living organism, comprising:
(a) dividing P₁′(t), which is obtained by differentiating one signal function P₁(t) between two signal functions having different wavelength bands and representing the physiological characteristics of the living organism, by P₂′(t) obtained by differentiating the other signal function P₂(t);
(b) obtaining a constant b of the following function n₁(t) and a cycle of the following function s₁(t) through Math Formula 1; and
(c) extracting a reference waveform of the function s₁(t) through Math Formula 4.
(P₁′(t)/P₂′(t))|_{t=t1,t2,t3, . . .} =k, [Math Formula 1]
(where P₁(t)=s₁(t)+n₁(t), P₂(t)=s₂(t)+n₂(t), as₁(t)=s₂(t), bn₁(t)=n₂(t), a, b and k are constants, t is time, and n₁(t) and n₂(t) are signal functions due to noise when measuring the physiological signal)
P₁(t)−P₂(t)/b=s₁(t)+n₁(t)−(as₁(t)+bn₁(t))/b=(1−a/b)s₁(t) [Math Formula 4]

10. The method according to claim 2, wherein Math Formula 1 used in extracting the constant b of the function n₁(t) and the cycle of the function s₁(t) is obtained based on that s₁′(t) obtained by differentiating the function s₁(t) is 0 (zero).

11. The method according to claim 3, wherein Math Formula 1 used in extracting the constant b of the function n₁(t) and the cycle of the function s₁(t) is obtained based on that s₁′(t) obtained by differentiating the function s₁(t) is 0 (zero).

12. The method according to claim 4, wherein Math Formula 1 used in extracting the constant b of the function n₁(t) and the cycle of the function s₁(t) is obtained based on that s₁′(t) obtained by differentiating the function s₁(t) is 0 (zero).

13. The method according to claim 5, wherein Math Formula 1 used in extracting the constant b of the function n₁(t) and the cycle of the function s₁(t) is obtained based on that s₁′(t) obtained by differentiating the function s₁(t) is 0 (zero).
